(19) United States
(12) Patent Application Publication
(10) Pub. No.: US 2004/0097465 A1
(43) Pub. Date: May 20, 2004

(54) ll-12 expression controlling agents

(76) Inventors: Akira Asari, Saitama (JP); Hitoshi Kurihara, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/471,824

(22) PCT Filed: Mar. 14, 2002
(86) PCT No.: PCT/JP02/02433

(30) Foreign Application Priority Data
Mar. 15, 2001 (JP) 2001-074077
Mar. 15, 2001 (JP) 2001-074078

(51) Int. Cl. A61K 31/728

(52) U.S. Cl. 514/54; 536/53

(57) ABSTRACT

An object of the present invention is to provide an IL-12 expression controlling agent, an inhibitor of IL-12 expression and an enhancer of IL-12 expression for oral administration, which comprise hyaluronan as an active ingredient. The present invention relates to an interleukin-12 expression controlling agent, which comprises hyaluronan as an active ingredient. Specifically, it is an inhibitor of interleukin-12 expression, which comprises hyaluronan or the pharmaceutically acceptable salt thereof having a weight average molecular weight of from 600,000 to 3,000,000 as an active ingredient, or an enhancer of interleukin-12 expression for oral administration, which comprise hyaluronan or the pharmaceutically acceptable salt thereof having a weight average molecular weight of from 50,000 to 400,000 as an active ingredient.

### TECHNICAL FIELD

The present invention relates to an interleukin 12 (IL-12) expression controlling agent, which comprises hyaluronan or a pharmaceutically acceptable salt thereof as an active ingredient.

Furthermore, the present invention relates to an inhibitor of interleukin 12 (IL-12) expression, which comprises hyaluronan or a pharmaceutically acceptable salt thereof having a weight average molecular weight of from 600,000 to 3,000,000 as an active ingredient.

Moreover, the present invention relates to an enhancer of interleukin 12 (IL12) expression for oral administration, which comprises hyaluronan or a pharmaceutically acceptable salt thereof having a weight average molecular weight of from 50,000 to 400,000 as an active ingredient.

Also, the present invention relates to an interleukin 12 (IL-12) expression controlling agent for oral administration, which comprises hyaluronan or a pharmaceutically acceptable salt thereof as an active ingredient.

### BACKGROUND OF THE INVENTION

It is known that IL-12 is a cytokine of a 70 kD glycoprotein (p70) in which two polypeptide chains of 35 kD (p35) and 40 kD (p40) are linked to each other, and plays a central role in the regulation of immunological functions in the living body (Cytokine, edited by Shinpei Kasakura, revised new edition of the Second Edition, pp. 207-225, published by Nihon Igakukan, Inc., Jun. 29, 1997).

It is known that since IL-12 acts on the differentiation induction of T helper 1 cell subset (Th1) of helper T cell, it acceleratively acts on the advance of morbid states in autoimmune diseases relating to activation of Th1.

J. Immunol., 165(4), 1863-1870 (2000) discloses that an oligosaccharide of hyaluronan increases IL-12 production of dendritic cells, but hyaluronan having a molecular weight of from 80,000 to 200,000 or a molecular weight of from 1,000,000 to 600,000 does not have such an activity. However, it neither discloses nor suggests the activity of hyaluronan to inhibit IL-12 production.

On the other hand, it is known that IL-12 plays a preventive role against microbial infection and shows an antitumor effect as a cytokine relating to the differentiation induction and the like of T helper 1 cell subset (Th1) of helper T cell.

J. Immunol., 159(5), 2492-2500 (1997) discloses that hyaluronan having a molecular weight of about 280,000 induced IL-12 production of macrophage in vitro.

Contrary to this, J. Immunol., 165(4), 1863-1870 (2000) discloses that an oligosaccharide of hyaluronan (about from 4 sugars to 14 sugars) increases IL-12 production of dendritic cells, but hyaluronan having a molecular weight of from 80,000 to 200,000 or a molecular weight of from 1,000,000 to 600,000 does not have such an activity.

Thus, various in vitro tests have been carried out regarding the acceleration of IL-12 production by hyaluronan, but information about which molecular weight of hyaluronan is effective for the acceleration of IL-12 production varies depending on literatures and is still unclear. In addition, all of these literatures are in vitro approach, and there is no approach actually carried out by taking note of its application as medicaments, particularly a “medicament for oral administration” which can be administered most conveniently.

An object of the present invention is to provide an IL-12 expression controlling agent, an inhibitor of IL-12 expression, an enhancer of IL-12 expression for oral administration and an IL-12 expression controlling agent for oral administration, which contain hyaluronan as an active ingredient.

### DISCLOSURE OF THE INVENTION

As a result of intensive studies carried out in order to solve the problems, the present inventors have found that hyaluronan controls IL-12 expression, specifically, hyaluronan having a specific weight average molecular weight has an activity to inhibit IL-12 expression, and that an inhibitor of IL-12 expression can be provided by using it. Also, the inventors have found that the above effect can also be obtained by oral administration of hyaluronan.

Furthermore, the inventors have found that hyaluronan having a specific weight average molecular weight has an activity to enhance IL-12 expression, and that the activity can also be obtained by oral administration to thereby find that an enhancer of IL-12 expression for oral administration can be provided by using it. The present invention has been completed based on these findings.

That is, the present invention provides an IL-12 expression controlling agent, which comprises hyaluronan or a pharmaceutically acceptable salt thereof as an active ingredient (hereinafter referred to as “controlling agent of the invention”). The controlling agent of the invention can inhibit or enhance IL-12 expression.

Furthermore, the present invention provides an inhibitor of IL-12 expression, which comprises hyaluronan or a pharmaceutically acceptable salt thereof having a weight average molecular weight of from 600,000 to 3,000,000 as an active ingredient (hereinafter referred to as “inhibitor of the invention”).

The weight average molecular weight of the hyaluronan or the pharmaceutically acceptable salt thereof used in the inhibitor of the invention is from 600,000 to 3,000,000, preferably from 600,000 to 1,200,000, more preferably from 700,000 to 1,200,000, still more preferably from 700,000 to 1,100,000, still far more preferably from 800,000 to 1,100,000, particularly preferably from 800,000 to 1,000,000, most preferably from 800,000 to 950,000, and far most preferably from 800,000 to 900,000.

The inhibitor of the invention can be used in all applications which require inhibition of IL-12 production. For example, it can be used as a reagent for inhibiting IL-12 expression in cells and tissues, and also as a medicament, food or the like for diseases in which inhibition of IL-12 expression in the living body is required.

It is preferable that the inhibitor of the invention is used for oral administration.

Furthermore, the present invention provides an enhancer of IL-12 expression for oral administration, which comprises hyaluronan or a pharmaceutically acceptable salt thereof having a weight average molecular weight of from 50,000 to 400,000 as an active ingredient (hereinafter referred to as “enhancer of the invention”).

The weight average molecular weight of the hyaluronan or the pharmaceutically acceptable salt thereof used in the enhancer of the invention is from 50,000 to 400,000, and is preferably from 100,000 to 300,000.

The enhancer of the invention can also be used as an oral administration medicament, food or the like for diseases in which enhancement of IL-12 expression in the living body is required.

In addition, the present invention provides an IL-12 expression controlling agent for oral administration, which comprises hyaluronan or a pharmaceutically acceptable salt thereof as an active ingredient (hereinafter referred to as “oral agent of the invention”). The oral agent of the invention can inhibit or enhance IL-12 expression by oral administration.

### BRIEF DESCRIPTION OF THE DRAWINGS

FIG. 1 shows decrease in serum IL-12 by oral administration of HA.

FIG. 2 shows increase in serum IL-12 by oral administration of HA.

### BEST MODE FOR CARRYING OUT THE INVENTION

### <1> Hyaluronan or Pharmaceutically Acceptable Salt Thereof

The origin of the hyaluronan or the pharmaceutically acceptable salt thereof used in the present invention is not particularly limited, and hyaluronan separated and purified from crests, umbilical cords, hyaluronan-producing microorganisms and the like can be used. Particularly, a preparation which is purified to such a high purity that it is substantially free of a substance whose contamination is not allowed as medicaments and food is preferred.

Examples of the pharmaceutically acceptable salt of hyaluronan include pharmaceutically acceptable salts among salts with inorganic base salts such as alkali metal salts (sodium salt, lithium salt, potassium salt, etc.), alkaline earth metal salts and ammonium salts and the like, and salts with organic bases such as diethanolamine salts, cyclohexylamine salts and amino acid salts. Among these, sodium hyaluronate is preferable.

When the controlling agent of the invention is used for inhibition of IL-12 expression, the following description on the inhibitor of the invention is similarly applied. Also, when it is used for enhancement of IL-12 expression, the following description on the enhancer of the invention is similarly applied. Accordingly, when the controlling agent of the invention is used for inhibition of IL-12 expression, the following description on the inhibitor of the invention should be referred, and when the controlling agent of the invention is used for the enhancement of IL-12 expression, the following description on the enhancer of the invention should be referred.

In addition, when the oral agent of the invention is used for inhibition of IL-12 expression, the following description on the inhibitor of the invention (a description part regarding oral administration) is similarly applied. Also, when it is used for enhancement of IL-12 expression, the following description on the enhancer of the invention (a description part regarding oral administration) is similarly applied. Accordingly, when the oral preparation of the present invention is used for inhibition of IL-12 expression, the following description on the inhibitor of the invention (a part regarding oral administration) should be referred, and when the controlling agent of the invention is used for the enhancement of IL-12 expression, the following description on the enhancer of the invention (a part regarding oral administration) should be referred.

The weight average molecular weight of the hyaluronan or the pharmaceutically acceptable salt thereof used in the inhibitor of the invention is not particularly limited, so long as it is from 600,000 to 3,000,000. As is shown below in Examples, hyaluronan or a pharmaceutically acceptable salt thereof shows excellent activity for production of IL-12 when the weight average molecular weight is about from 840,000 to 850,000, and shows effects within the above specific weight average molecular weight range. The lower limit of the weight average molecular weight of the hyaluronan or the pharmaceutically acceptable salt thereof used in the inhibitor of the invention is 600,000, preferably 700,000, more preferably 800,000, and the upper limit thereof is 3,000,000, preferably 1,200,000, more preferably 1,100,000, still more preferably 1,000,000, still far more preferably 950,000, and particularly preferably 900,000. The weight average molecular weight of the hyaluronan or the pharmaceutically acceptable salt thereof used in the inhibitor of the invention is most preferably about from 840,000 to 900,000.

The hyaluronan or the pharmaceutically acceptable salt thereof used in the inhibitor of the invention has a limiting viscosity of about from 11.5 to 44 dl/g, preferably about from 11.5 to 20 dl/g, more preferably about from 13.0 to 20 dl/g, still more preferably about from 13.0 to 18.5 dl/g, still far more preferably about from 14.5 to 18.5 dl/g, particularly preferably about from 14.5 to 17.5 dl/g, most preferably about from 14.5 to 16.5 dl/g, far most preferably about from 14.5 to 16 dl/g, ultimately preferably about from 15 to 16 dl/g. Among these, one having a limiting viscosity of around 15 dl/g is more preferable.

An inhibitor of IL-12 expression having excellent activity can be obtained by using the above hyaluronan or pharmaceutically acceptable salt thereof On the other hand, the weight average molecular weight of the hyaluronan or the pharmaceutically acceptable salt thereof used in the enhancer of the invention is not particularly limited, so long as it is from 50,000 to 400,000. As is described below in Examples, hyaluronan or a pharmaceutically acceptable salt thereof shows excellent activity for IL-12 production when the weight average molecular weight is from 100,000 to 300,000, and shows the effect within the above specific weight average molecular weight range.

The lower limit of the weight average molecular weight of the hyaluronan or the pharmaceutically acceptable salt thereof used in the enhancer of the invention is 50,000 and preferably 100,000, and the upper limit thereof is 400,000 and preferably 300,000.

The above hyaluronan or pharmaceutically acceptable salt thereof used in the enhancer of the invention has a limiting viscosity of about from 2 to 8.5 dl/g, and preferably about from 5 to 7 dl/g.

An enhancer of IL-12 expression for oral administration having excellent activity can be obtained by using the above hyaluronan or pharmaceutically acceptable salt thereof.

Also, the weight average molecular weight of the hyaluronan or the pharmaceutically acceptable salt thereof used in the inhibitor of the invention or the enhancer of the invention can be calculated based on the equation of Laurent et al. (Biochim. Biophys. Acta, 42, 476 (1960)) by measuring its limiting viscosity in accordance with The Pharmacopoeia of Japan, 13th revised edition: General Test Method, Chapter 36, Viscosity Measuring Method.

Also the upper limit of the endotoxin concentration in the hyaluronan or the pharmaceutically acceptable salt thereof used in the inhibitor of the invention and the enhancer of the invention can be optionally set according to specific use and the like of the inhibitor of the invention and the enhancer of the invention. For example, when it is used as a reagent for use in a test which should be carried out in the absence of an endotoxin or as a medicament or the like which is directly administered into blood vessels, it is preferably 0.3 EU/ml or less when it is made into a solution preparation. Also, when orally administered like the case of the enhancer of the invention, it can be optionally set within such a range that it does not cause problems in carrying out oral administration. In this case, it is preferably 0.3 EU/ml or less when it is made into a solution preparation.

The endotoxin concentration can be measured using any endotoxin measuring method well known to and conventionally used by one skilled in the art, but the Limulus test using a horseshoe crab amoebocyte lysate component is preferable. Also, the EU (endotoxin unit) can be measured and calculated in accordance with Japan Industrial Standard, Provisions for Biochemical Reagents (JIS K8008). In addition, it is preferable that the iron content is 20 ppm or less.

### <2> Dosage Form, Etc. of the Inhibitor of the Invention or the Enhancer of the Invention

An applying method of the inhibitor of the invention to cells and tissues which require inhibition of IL-12 production is not particularly limited, too, so long as the IL-12 expression inhibiting effect by the inhibitor of the invention is obtained, and it can be optionally selected according to specific use and the like of the inhibitor of the invention. For example, when it is used as a reagent for testing cells and tissues, the inhibitor of the invention may be added to a culture medium or the like to culture cells or tissues using it, or the inhibitor of the invention may be directly added to cells or tissues.

When the inhibitor of the invention is used as a medicament or the like, it can be administered, for example, by a method such as injection (intravenous, intramuscular, subcutaneous, intracutaneous, intraperitonealm, etc.), nasal administration, oral administration, percutaneous administration or inhalation. According to these administration methods, it can be prepared as injections (solutions, suspensions, emulsions, solid preparations for dissolution before use, etc.), tablets, capsules, solutions, granules, powders, lipo-forming preparations, ointments, plasters, lotions, pastes, adhesive preparations, gels, suppositories, powders for external use, sprays, inhalation powders and the like.

The concentration of the hyaluronan or the pharmaceutically acceptable salt thereof in the inhibitor of the invention is not particularly limited, too, and is preferably from 0.5 to 10% (w/v). Particularly, when the inhibitor of the invention is provided as injections, the concentration is preferably about from 1 to 5% (w/v), more preferably about from 1 to 3% (w/v), and most preferably about from 1 to 2% (w/v). In addition, when the inhibitor of the invention is used as a preparation for oral administration, for example as solutions, the concentration is preferably 0.1% (w/v) or more, and more preferably about from 0.1 to 1% (w/v).

On the other hand, the enhancer of the invention is orally administered for applying to an animal which requires enhancement of IL-12 production. According to the object, subject and the like of oral administration, the enhancer of the invention can be prepared as tablets, capsules, solutions, granules, powders, lipo-forming preparations, inhalation powders and the like.

The concentration of the hyaluronan or the pharmaceutically acceptable salt thereof in the enhancer of the invention is not particularly limited, too, and is preferably from 0.5 to 10% (w/v). For example, when the enhancer of the invention is used as solutions for oral administration, the concentration is preferably 0.5% (w/v) or more, and more preferably about from 0.5 to 2% (w/v).

The inhibitor of the invention or the enhancer of the invention can be prepared using known methods. In addition, in preparing these preparations, other pharmaceutically active components and components generally used in medicaments, such as conventionally used stabilizing agents, emulsifying agents, osmotic pressure adjusting agents, buffer agents, tonicity agents, preservatives, soothing agents, coloring agents, excipients, binders, lubricants, disintegrating agents and the like, can be used, so long as they do not show bad influences on the hyaluronan or the pharmaceutically acceptable salt thereof and also do not show influences on the effects of the present invention.

### <3> Subject of Administration, Etc. when the Inhibitor of the Invention or the Enhancer of the Invention is Used for Medical Treatment

As the animal to which the inhibitor of the invention is administered, vertebral animals, particularly mammals, are preferable, and human is particularly preferable. The inhibitor of the invention can be administered to these animals as a medicament for the purpose of inhibiting IL-12 production. The applicable disease is not limited, so long as the inhibition of IL-12 production is the object, and examples include diseases whose cause of disease is the activation of Th1 in which IL-12 positively acts upon the advance of the condition of the disease. Specific examples of the diseases include contact dermatitis, autoimmune uvea retinitis, allergic cerebrospinal meningitis, insulin-dependent diabetes mellitus, diabetes mellitus, Hashimoto disease, multiple sclerosis, rheumatic arthritis, Sjögren syndrome, Crohn disease, sarcoidosis, psoriasis, lipopolysaccharide-induced hepatic necrosis, crescentic glomerulonephritis, systemic lupus erythematosus and the like. Thus, the inhibitor of the invention also includes a concept as a treating agent of these diseases. When the inhibitor of the invention is used as a medicament, its administration may be not only for the purpose of pure treatment but also for the purpose of prevention, advance inhibition (worsening prevention), alleviation (improvement of symptoms) and the like of diseases.

A formulating amount, a dose per one administration, an administration interval and the like of the hyaluronan or the pharmaceutically acceptable salt thereof in the inhibitor of the invention are not particularly limited, because they are items which should be separately decided according to the administration method, dosage form, using purpose and the like of the inhibitor of the invention and specific symptoms, age, sex, body weight and the like of each patient, but as the clinical dose of the hyaluronan or a pharmaceutically acceptable salt thereof, about from 5 to 25 mg per once and from 10 to 50 mg per day, per adult can be exemplified in the case of parenteral administration by injection, and from 50 to 250 mg per once and from 100 to 500 mg per day, per adult in the case of oral administration. Also, the administration interval of the inhibitor of the invention may be about once a day, and it can be administered by dividing the daily dose into 2 to 3 doses per day. Alternatively, it may be administered once during a period of about from 1 to 3 days.

As the animal to which the enhancer of the invention is administered, vertebral animals, particularly mammals, are also preferable, and human is particularly preferable. The enhancer of the invention can be orally administered to these animals as a medicament for the purpose of enhancing IL-12 production. The applicable disease is not limited, so long as the enhancement of IL-12 production is the object, and examples include diseases caused by microbial infection, viral diseases (AIDS, hepatitis C and the like), tumors (cancers), diseases caused by the reduction of IL-12 production, and the like. Thus, the enhancer of the invention also includes a concept as a treating agent of these diseases. When the enhancer of the invention is used as a medicament, its administration may be not only for the purpose of pure treatment but also for the purpose of prevention, advance inhibition (worsening prevention), alleviation (improvement of symptoms) and the like of diseases.

A formulating amount, a dose per one administration, an administration interval and the like of the hyaluronan or the pharmaceutically acceptable salt thereof in the enhancer of the invention are not particularly limited, because they are items which should be separately decided according to the administration method, dosage form, using purpose and the like of the enhancer of the invention and specific symptoms, age, sex, body weight and the like of each patient, but as the clinical dose of the hyaluronan or a pharmaceutically acceptable salt thereof, about from 5 to 25 mg per once and from 10 to 50 mg per day, per adult can be exemplified in the case of parenteral administration by injection, and from 50 to 250 mg per once and from 100 to 500 mg per day, per adult in the case of oral administration. Also, the administration interval of the enhancer of the invention may be about once a day, and it can be administered by dividing the daily dose into 2 to 3 doses per day. Alternatively, it may be administered once during a period of about from 1 to 3 days.

Also, hyaluronan or pharmaceutically acceptable salts thereof have already been used in medicaments, cosmetics, food and the like and it is known that they have high safety.

### EXAMPLES

Examples of the present invention are specifically described below. However, technical scope of the present invention is not limited thereto.

### Example 1

### Inhibition of IL-12 Expression

### <1> Material to be Tested

The materials to be tested used in this example are as follows.

Phosphate buffered saline (PBS)

Sodium hyaluronate (weight average molecular weight: 840,000; limiting viscosity: 15.0 dl/g). Hereinafter, this sodium hyaluronate is referred to as HA.

HA was used by dissolving it in PBS to a predetermined concentration according to each of the following drug efficacy pharmacological tests. The endotoxin concentration after dissolving in PBS was 0.3 EU/ml or less in each case, and the iron content was 20 ppm or less in each case.

### <2> Drug Efficacy Pharmacological Test

### (1) Reduction of [IL-12 p40 Production in Macrophage

Into the abdominal cavity of a 4-weeks-old male C57BL/6 mouse (Charles River Japan, Inc.), 5 to 7 ml of ice-cooled Hanks' solution was injected and recovered 5 minutes thereafter to collect commonly existing peritoneal cells. The thus collected cells were dispensed in a 96 well flat bottom plate and cultured using DMEM medium at 37° C. overnight under 5% CO₂. After the culturing, suspended cells were removed and the adhered cells were used in the test as the peritoneal macrophage.

In order to stimulate IL-12 production, a lipopolysaccharide (LPS) (List Biological Laboratories, Inc.) was added to media containing varied concentrations of HA, to give a final concentration of 1 μg/ml, and the cells were treated for 24 hours. Thereafter, the concentration of IL-12 (p40) in the cell culture supernatant was measured using Mouse IL-12 (p40) ELISA Kit (manufactured by Endogen) to evaluate IL-12 production. In this case, the test was carried out three times and the average value was calculated. The results are shown below.

TABLE 1

| No treatment | LPS alone | LPS + 10 μg/ml HA | LPS + 100 μg/ml HA |
|---|---|---|---|
| 0.0 | 26.8 | 21.3 | 18.5 |

(unit: pg/ml)

Based on this result, it was shown that a high molecular weight sodium hyaluronate having a weight average molecular weight of about 840,000 inhibits IL-12 production.

Separately from this, a similar test was carried out using sodium hyaluronate (100 μg/ml) having other weight average molecular weight (600,000 or 2,700,000; limiting viscosity 11.5 dl/g or 40.5 dl/g). The test was carried out three times and the average value was calculated.

As a result, the concentration of IL-12 (p40) in the cell culture supernatant was 65.1 pg/ml (4.6) in the case of LPS alone, 56.0 pg/ml (5.1) in the case of LPS+ sodium hyaluronate having a weight average molecular weight of 600,000, and 60.0 pg/ml (7.7) in the case of LPS +sodium hyaluronate having a weight average molecular weight of 2,700,000. Also, the numeral in parentheses indicates a standard deviation.

Based on this result, each of the sodium hyaluronate samples did not inhibit IL-12 production significantly but showed a tendency to inhibit it though slightly. In addition, this tendency was slightly high in the case of the molecular weight of 600,000.

### (2) Reduction of Serum IL-12 by HA Administration

Four-weeks-old male C57BL/6 mice of 4 weeks of age (Charles River Japan, Inc.) were purchased and divided into PBS administration group (n=5) and HA administration group (n=5).

A PBS solution containing 10 mg/ml HA was orally administered at a dose of 80 mg/kg body weight per once. The oral administration was carried out in the usual way using sterilized 1 ml capacity disposable syringes and sterilized oral sounds. The administration was carried out twice a day continuously for 2 weeks.

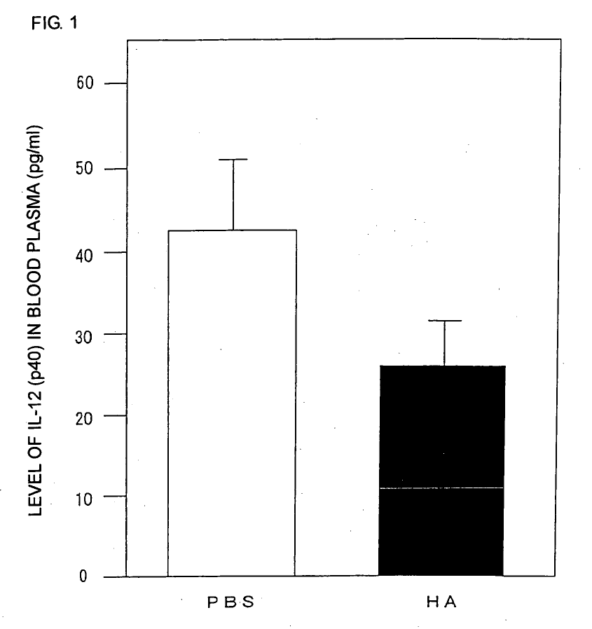

After completion of the administration, blood samples were collected from the orbital venous plexus, and concentration of IL-12 (p40) in blood plasma was measured using Mouse IL-12 (p40) ELISA Kit (manufactured by Endogen) to calculate its average value and standard deviation. The results are shown in FIG. 1.

Based on this result, it was shown that IL-12 production can be inhibited also by oral administration of a high molecular weight sodium hyaluronate having a weight average molecular weight of about 840,000.

### Example 2

### Enhancement of IL-12 Expression

### <1> Material to be Tested

The materials to be tested used in this example are as follows.

Phosphate buffered saline (PBS)

Sodium hyaluronate (weight average molecular weight: 100,000 to 200,000; limiting viscosity: 3 dl/g to 5 dl/g). Hereinafter, this sodium hyaluronate is called “HA10-20”.

Sodium hyaluronate (weight average molecular weight: about 300,000; limiting viscosity: about 7 dl/g). Hereinafter, this sodium hyaluronate is called “HA30”.

HA was used by dissolving it in PBS to a predetermined concentration according to each of the following drug efficacy pharmacological tests. The endotoxin concentration after dissolving in PBS was 0.3 EU/ml or less in each case, and the iron content was 20 ppm or less in each case.

### <2> Drug Efficacy Pharmacological Test

### (1) Increase of Serum IL-12 by HA10-20 Administration

Four-weeks-old male C57BL/6 mice (Charles River Japan, Inc.) were purchased and divided into PBS administration group (n=5) and HA10-20 administration group (n=5).

A PBS solution containing 10 mg/ml HA10-20 was orally administered at a dose of 80 mg/kg body weight per once. The oral administration was carried out in the usual way using sterilized 1 ml capacity disposable syringes and sterilized oral sounds. The administration was carried out twice a day continuously for 2 weeks.

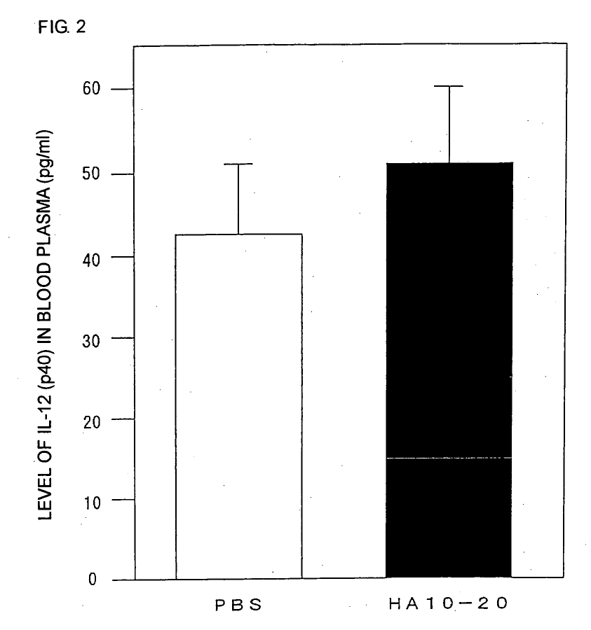

After completion of the administration, blood samples were collected from the orbital venous plexus, and concentration of IL-12 (p40) in blood plasma was measured using Mouse IL-12 (p40) ELISA Kit (manufactured by Endogen) to calculate its average value and standard deviation. The results are shown in FIG. 2.

Based on this result, it was shown that IL-12 production can be enhanced by oral administration of a low molecular weight sodium hyaluronate having a weight average molecular weight of about from 100,000 to 200,000.

### (2) Increase of Serum IL-12 by HA30 Administration

Four-weeks-old male C57BL/6 mice (Charles River Japan, Inc.) were purchased and divided into PBS administration group (n=5) and HA30 administration group (n=5).

A PBS solution containing 100 μg/ml HA30 was orally administered at a dose of 0.5 ml (about 2.5 mg/kg body weight) per once. The oral administration was carried out in the usual way similar to the above case. The administration was carried out once a day continuously for 13 days.

After completion of the administration, blood samples were collected from the orbital venous plexus, and concentration of IL-12 (p40) in blood plasma was measured by the same method described above to calculate its average value and standard deviation. The result (average value±standard deviation) is shown below.

PBS administration group: 151.5±18.4 (pg/ml)

HA30 administration group: 212.8±23.6 (pg/ml)

In addition, this result was significant with p<0.05 by Student's T-test.

Based on the above result, it was shown that IL-12 production can be significantly enhanced by oral administration of a low molecular weight sodium hyaluronate having a weight average molecular weight of about 300,000.

Thus, regarding the IL-12 production, the hyaluronan having a molecular weight of 600,000 showed a tendency to inhibit it slightly, the hyaluronan having a molecular weight of 840,000 inhibited it considerably, and the hyaluronan having a molecular weight of 2,700,000 showed a tendency to inhibit it though very slightly. On the other hand, the hyaluronan having a molecular weight of about from 100,000 to 300,000 conversely enhanced the IL-12 production. When these results are synthetically taken into consideration, it is considered that hyaluronan exerts the action to inhibit IL-12 expression within a range of molecular weight centering at 840,000 and extending plus and minus sides than that by a factor of about 300,000 (a molecular weight of from 600,000 to 1,200,000).

While the invention has been described in detail and with reference to specific embodiments thereof, it will be apparent to one of skill in the art that various changes and modifications can be made therein without departing from the spirit and scope thereof.

This application is based on Japanese application No. 2001-074077 filed on Mar. 15, 2001 and Japanese application No. 2001-074078 filed on Mar. 15, 2001, the entire contents of which are incorporated hereinto by reference.

### Industrial Applicability

The controlling agent of the invention comprising hyaluronan or a pharmaceutically acceptable salt thereof as an active ingredient is markedly useful for the treatment of cells and tissues which require control of IL-12 expression (inhibition or enhancement of expression).

Since the inhibitor of the invention comprising a high molecular weight hyaluronan having a specified weight average molecular weight or a pharmaceutically acceptable salt thereof as an active ingredient shows an effect to inhibit IL-12 production as is apparent also from the results of the above drug efficacy pharmacological tests, it is markedly useful for the treatment of cells and tissues which require inhibition of IL-12 production.

Particularly, since the inhibitor of the invention shows IL-12 expression inhibitory activity by its oral administration and a natural product is used as the material, its safety is also high and its availability is markedly high.

On the other hand, since the enhancer of the invention comprising a low molecular weight hyaluronan having a specified weight average molecular weight or a pharmaceutically acceptable salt thereof as an active ingredient shows an effect to enhance IL-12 production by its oral administration as is apparent also from the results of the above drug efficacy pharmacological tests, it is markedly useful for the treatment of animals which require enhancement of IL-12 production.

Particularly, since the enhancer of the invention uses a natural product as the material, its safety is also high and its availability is markedly high.

In addition, since the oral preparation of the present invention can be conveniently administered to patients by its oral administration, it is markedly useful.

1. An interleukin-12 expression controlling agent, which comprises hyaluronan or a pharmaceutically acceptable salt thereof as an active ingredient.

2. An inhibitor of interleukin-12 expression, which comprises hyaluronan or a pharmaceutically acceptable salt thereof having a weight average molecular weight of from 600,000 to 3,000,000 as an active ingredient.

3. The inhibitor according to claim 2, wherein the hyaluronan or the pharmaceutically acceptable salt thereof has a weight average molecular weight of from 600,000 to 1,200,000.

4. The inhibitor according to claim 2, wherein the hyaluronan or the pharmaceutically acceptable salt thereof has a weight average molecular weight of from 700,000 to 1,200,000.

5. The inhibitor according to claim 2, wherein the hyaluronan or the pharmaceutically acceptable salt thereof has a weight average molecular weight of from 700,000 to 1,100,000.

6. The inhibitor according to claim 2 or 3, wherein the hyaluronan or the pharmaceutically acceptable salt thereof has a weight average molecular weight of from 800,000 to 1,100,000.

7. The inhibitor according to claim 2 or 3, wherein the hyaluronan or the pharmaceutically acceptable salt thereof has a weight average molecular weight of from 800,000 to 1,000,000.

8. The inhibitor according to claim 2 or 3, wherein the hyaluronan or the pharmaceutically acceptable salt thereof has a weight average molecular weight of from 800,000 to 950,000.

9. The inhibitor according to claim 2 or 3, wherein the hyaluronan or the pharmaceutically acceptable salt thereof has a weight average molecular weight of from 800,000 to 900,000.

10. The inhibitor according to any one of claims 2 to 9, which is used for oral administration.

11. An enhancer of interleukin-12 expression for oral administration, which comprises hyaluronan or a pharmaceutically acceptable salt thereof having a weight average molecular weight of from 50,000 to 400,000 as an active ingredient.

12. The enhancer according to claim 11, wherein the hyaluronan or the pharmaceutically acceptable salt thereof has a weight average molecular weight of from 100,000 to 300,000.

13. An interleukin-12 expression controlling agent for oral administration, which comprises hyaluronan or a pharmaceutically acceptable salt thereof as an active ingredient.
